(19) United States
(12) Patent Application Publication
(10) Pub. No.: US 2024/0100245 A1
(43) Pub. Date: Mar. 28, 2024

(54) Wearable Medical Infusion Pump and Method

(71) Applicant: Becton, Dickinson and Company, Franklin Lakes, NJ (US)

(72) Inventor: Kevin M. Ryan, Whitehouse Station, NJ (US)

(21) Appl. No.: 17/950,474

(22) Filed: Sep. 22, 2022

(51) Int. Cl. A61M 5/142; A61M 5/145

(52) U.S. Cl. CPC A61M 5/14212; A61M 5/145; A61M 2005/1403

(57) ABSTRACT

A pump for delivering fluid from a reservoir to a patient includes at least one pumping syringe having a barrel and a plunger movable through the barrel and at least one actuator for moving the plunger through the barrel to draw the fluid from the reservoir into the barrel and to expel the fluid from the barrel towards the patient. The pump also includes at least one inflow valve between the reservoir and the barrel of the at least one pumping syringe that opens when the plunger moves to aspirate the fluid from the reservoir into the barrel; and at least one outflow valve between the barrel of the at least one pumping syringe and the patient that opens when the plunger moves to expel the fluid from the barrel of the at least one pumping syringe towards the patient.

### BACKGROUND OF THE INVENTION

### Field of the Invention

The present invention relates generally to wearable injection and/or infusion devices and, in particular, to a wearable pump for administrating a medical fluid, such as saline, a therapeutic agent, and/or total parenteral nutritional fluid, to a patient as a continuous, substantially continuous, semi-continuous, or intermittent fluid flow.

### Description of the Related Art

Various types of automatic injection devices have been developed to allow drug solutions and other liquid therapeutic preparations to be administered by untrained personnel or for self-injection. Generally, these devices include a reservoir that is pre-filled with the liquid therapeutic preparation and some type of automatic needle-injection mechanism that can be triggered by the user. When the volume of fluid or drug to be administered is above 1 mL, the injection time generally becomes longer making it more difficult for the patient to maintain contact between the device and a target area of the patient's skin. Further, as the volume of drug to be administered becomes larger, it may be desirable to increase a time period for performing the injection. A traditional method for slowly injecting a therapeutic agent to a patient is to use an intravenous (IV) bag, where fluid slowly passes from the bag through a fluid conduit and to the patient by gravity. Such a procedure is typically performed in a hospital or outpatient setting because the IV bag is often supported by a vertical pole or similar support so that the IV bag remains above the injection site allowing the medical fluid to drain from the bag toward the patient by gravity.

Certain devices allow for self-injection or self-infusion in a home setting and are capable of gradually injecting a liquid therapeutic preparation into the skin of a patient. In some cases, these devices are small enough (both in height and in overall size) to allow the devices to be “worn” by an ambulatory patient while the liquid therapeutic preparation is being infused into the patient. These wearable injection and/or infusion devices typically include a pump or another type of discharge mechanism to force a liquid therapeutic preparation to flow out of a reservoir and into an injection needle or vascular access set. Such devices also typically include a valve or flow control mechanism to cause the liquid therapeutic preparation to begin to flow at the proper time and at a selected flow rate, as well as an electrical and/or mechanical triggering mechanism to initiate the injection.

One type of wearable infusion pump is an elastomeric infusion bag. Such elastomeric infusion bags provide a continuous flow of fluid to the patient; however, the flow rate accuracy can be poor because elasticity of the bag material changes over time. Therefore, such elastomeric bags may become less accurate and/or may not function as anticipated after a preferred or recommended shelf life for the bag device expires. Changes in elasticity of the bag material may also cause changes to bag fill volume, as well as variations in IV catheter lumen size (inner diameter) and/or in IV line % blockage or flow restrictions.

Another type of wearable infusion pump is an electromechanical peristaltic pump. Peristaltic pumps typically have marginally better accuracy than elastomeric infusion bags. However, the peristaltic pumps have poor flow rate accuracy when compared to intravenous (IV) infusion syringe pumps. Therefore, infusion of medications that requires high accuracy may not be recommended with wearable peristaltic pumps.

IV syringe pumps, known in the prior art, are a third fluid delivery option. However, such IV syringe pumps have several draw-backs that may make them unsuitable for use as a wearable pump. For example, IV syringe pumps are typically designed to hold large syringe sizes, up to a 60 mL or 100 mL. In order to hold such large syringes, IV syringe pumps can be large in size and weight due to the linear actuator mechanisms that are needed to drive the large syringes. The large size and weight of the drive components can limit the pump to non-wearable bed-side or IV pole mounted use. Additionally, if the patient needs a larger volume of medication or medication to be infused for a long time period (several days), the syringes have to be frequently replaced over the infusion period. Using large volume syringes also leads to lower infusion volume accuracy due to manufacturing tolerances and other factors, as compared to pumps comprising smaller syringes or fluid reservoirs.

In view of the limits of currently available wearable pumps, there is a need for a wearable pump that is capable of more precise flow rate accuracy, while having the ability to pump large fluid volumes over extended periods of time. Also, pump drive components should be small enough to be wearable. There is also a need for a pump that is configured to provide a flow of fluid that is continuous, semi-continuous, or intermittent. The medical infusion pumps and associated methods of the present disclosure are configured to address such limitations of prior infusion pump designs.

### SUMMARY OF THE INVENTION

According to an aspect of the disclosure, a pump for delivering fluid from a reservoir to a patient includes at least one pumping syringe having a barrel and a plunger movable through the barrel, and at least one actuator for moving the plunger through the barrel to draw the fluid from the reservoir into the barrel and to expel the fluid from the barrel towards the patient. The pump also includes at least one inflow valve between the reservoir and the barrel of the at least one pumping syringe that opens when the plunger of the at least one pumping syringe moves to aspirate the fluid from the reservoir into the barrel of the at least one pumping syringe; and at least one outflow valve between the barrel of the at least one pumping syringe and the patient that opens when the plunger moves to expel the fluid from the barrel of the at least one pumping syringe towards the patient.

According to another aspect of the disclosure, a fluid infusion system includes: the previously-described pump; the reservoir fluidly connected to the pump containing the fluid to be delivered to the patient; and at least one patient line fluidly connected to the barrel of the at least one pumping syringe of the pump at a position distal to the at least one outflow valve. The patient line is configured to deliver the fluid from the pump to vasculature of the patient.

According to another aspect of the disclosure, a method for fluid infusion to a patient includes attaching the previously-described pump to the reservoir, such that the reservoir is in fluid communication with the barrel of the at least one pumping syringe of the pump. The method also includes placing the pump in fluid communication with vasculature of the patient. The method also includes causing the at least one actuator of the pump to repeatedly move the plunger through the barrel of the at least one pumping syringe, thereby causing the fluid to pass from the reservoir into the barrel of the at least one pumping syringe and from the barrel of the at least one pumping syringe to the patient.

Non-limiting illustrative examples of embodiments of the present disclosure will now be described in the following numbered clauses.

Clause 1: A pump for delivering fluid from a reservoir to a patient, comprising: at least one pumping syringe comprising a barrel and a plunger movable through the barrel; at least one actuator for moving the plunger through the barrel to draw the fluid from the reservoir into the barrel and to expel the fluid from the barrel towards the patient; at least one inflow valve between the reservoir and the barrel of the at least one pumping syringe that opens when the plunger of the at least one pumping syringe moves to aspirate the fluid from the reservoir into the barrel of the at least one pumping syringe; and at least one outflow valve between the barrel of the at least one pumping syringe and the patient that opens when the plunger moves to expel the fluid from the barrel of the at least one pumping syringe towards the patient.

Clause 2: The pump of clause 1, wherein the pump is a wearable pump worn by an ambulatory patient.

Clause 3: The pump of clause 1 or clause 2, wherein the fluid comprises at least one of saline, a liquid medication, or a total parenteral nutrition liquid.

Clause 4: The pump of any of clauses 1-3, wherein the at least one actuator comprises a linear actuator that engages the plunger to move the plunger through the barrel.

Clause 5: The pump of any of clauses 1-4, further comprising a valve set connected to the at least one pumping syringe and to the reservoir, the valve set comprising: a housing enclosing at least one fluid chamber; at least one inflow port, wherein the fluid passes from the reservoir to the at least one fluid chamber through the at least one inflow port; at least one syringe port fluidly connected to an interior of the barrel of the at least one pumping syringe, wherein the fluid passes between the interior of the barrel and the at least one fluid chamber through the at least one syringe port; and at least one outflow port, wherein the fluid passes from the at least one fluid chamber to the patient through the at least one outflow port.

Clause 6: The pump of clause 5, wherein the at least one inflow valve is integral with the at least one inflow port.

Clause 7: The pump of clause 5 or clause 6, wherein the at least one outflow valve is integral with the at least one outflow port.

Clause 8: The pump of any of clauses 5-7, wherein the at least one inflow port, the at least one syringe port, and/or the at least one outflow port are removably connected to the housing.

Clause 9: The pump of any of clauses 1-8, wherein the at least one inflow valve and the at least one outflow valve are one-way check valves.

Clause 10: The pump of any of clauses 1-9, wherein the at least one inflow valve comprises an inflow check valve that automatically opens as the fluid is aspirated into the barrel of the at least one pumping syringe.

Clause 11: The pump of clause 10, wherein the at least one outflow valve comprises an outflow check valve that automatically opens when the fluid is expelled from the barrel of the at least one pumping syringe.

Clause 12: The pump of clause 11, wherein a cracking pressure of the inflow check valve and/or the outflow check valve is greater than atmospheric pressure to prevent siphoning of the fluid from the reservoir through the inflow check valve and the outflow check valve.

Clause 13: The pump of any of clauses 1-12, wherein a volume of the barrel of the at least one pumping syringe is from about 3 mL to about 5 mL.

Clause 14: The pump of any of clauses 1-13, wherein the barrel of the at least one pumping syringe comprises an open proximal end, a distal end comprising a fluid port, and a cylindrical sidewall extending therebetween.

Clause 15: The pump of clause 14, wherein the plunger comprises an elastomeric stopper that seals against an inner surface of the cylindrical sidewall of the barrel and a plunger rod connected to a proximal end of the stopper for moving the stopper through the barrel to aspirate the fluid into the barrel through the fluid port and to expel the fluid from the barrel from the fluid port.

Clause 16: The pump of clause 15, wherein the at least one actuator engages a proximal end of the plunger rod and moves the plunger rod backwards through the barrel to aspirate the fluid into the barrel and forward through the barrel to expel the fluid from the barrel.

Clause 17: The pump of clause 16, wherein the actuator comprises a retainer that engages a proximal end of the plunger rod, and wherein the actuator moves the retainer back and forth along a track, thereby moving the elastomeric stopper through the barrel.

Clause 18: The pump of any of clauses 1-17, further comprising a pump controller electrically coupled to the at least one actuator configured to cause the at least one actuator to repeatedly move the plunger through the barrel of the at least one pumping syringe at a predetermined rate.

Clause 19: The pump of clause 18, wherein the pump controller is configured to monitor a torque exerted by the at least one actuator on the plunger to detect when the plunger approaches an end of stroke position.

Clause 20: The pump of clause 19, wherein the pump controller is configured to change direction of the plunger when the torque increases above a threshold.

Clause 21: The pump of any of clauses 18-20, further comprising at least one position sensor for detecting a position of the plunger within the barrel, wherein the pump controller is configured to cause the at least one actuator to change direction of the plunger based on information detected by the at least one position sensor.

Clause 22: The pump of any of clauses 19-21, wherein the pump controller is configured to receive an input from a user and adjust a rate at which the at least one actuator moves the plunger through the barrel based on the received input.

Clause 23: The pump of any of clauses 18-22, further comprising a bolus input button, and wherein the pump controller is configured to receive an indication when the bolus input button is pressed by the user, and cause the at least one actuator to increase a rate at which the plunger moves through the barrel for a predetermined duration to provide a bolus of the fluid to the patient.

Clause 24: The pump of any of clauses 1-23, wherein the reservoir comprises a reservoir syringe comprising a barrel and a stopper for retaining the fluid within the barrel.

Clause 25: The pump of clause 24, wherein the barrel of the reservoir syringe comprises an open proximal end, a distal end comprising a fluid port directly or indirectly connected to the at least one inflow valve, and a cylindrical sidewall extending between the proximal end and the distal end.

Clause 26: The pump of any of clauses 1-25, wherein the reservoir comprises an intravenous bag fluidly connected to the at least one inflow valve by at least one fluid conduit.

Clause 27: The pump of any of clauses 1-26, further comprising an enclosure that contains at least portions of the at least one pumping syringe, the reservoir, the at least one inflow valve, and the at least one outflow valve.

Clause 28: The pump of clause 27, wherein the enclosure comprises a sealed enclosure to restrict access to the fluid in the reservoir.

Clause 29: The pump of clause 27 or clause 28, wherein the enclosure comprises a clam-shell enclosure comprising a base and a cap pivotally connected to the base along a hinge.

Clause 30: The pump of any of clauses 1-29, configured to provide continuous, semi-continuous, or intermittent fluid delivery to an ambulatory patient.

Clause 31: The pump of any of clauses 1-30, wherein the reservoir is removable from other portions of the pump allowing the reservoir to be replaced without ceasing operation of the at least one actuator and the at least one pumping syringe.

Clause 32: The pump of any of clauses 1-31, wherein the at least one pumping syringe comprises a first pumping syringe and a second pumping syringe, and wherein the at least one actuator comprises a first actuator that moves the plunger of the first pumping syringe and a second actuator that moves the plunger of the second pumping syringe.

Clause 33: The pump of clause 32, wherein the fluid expelled from the first pumping syringe passes to a first patient line and fluid expelled from the second pumping syringe passes to a second patient line that is separate from the first patient line.

Clause 34: The pump of clause 32 or clause 33, wherein the fluid expelled from the first pumping syringe and the fluid expelled from the second pumping syringe pass to the patient through a common patient line.

Clause 35: The pump of any of clauses 32-34, wherein the at least one inflow valve comprises: a first inflow valve between the reservoir and the barrel of the first pumping syringe that opens when the plunger of the first pumping syringe moves to aspirate the fluid from the reservoir into the barrel of the first pumping syringe; and a second inflow valve between the reservoir and the barrel of the second pumping syringe that opens when the plunger of the second pumping syringe moves to aspirate the fluid from the reservoir into the barrel of the second pumping syringe.

Clause 36: The pump of clause 35, wherein the first inflow valve closes when the plunger of the first pumping syringe moves to expel the fluid from the barrel of the first pumping syringe, and the second inflow valve closes when the plunger of the second pumping syringe moves to expel the fluid from the barrel of the second pumping syringe.

Clause 37: The pump of clause 35 or clause 36, wherein the at least one outflow valve comprises a first outflow valve between the barrel of the first pumping syringe and the patient that opens when the plunger of the first pumping syringe moves to expel the fluid from the barrel towards the patient, and a second outflow valve between the barrel of the second pumping syringe and the patient that opens when the plunger of the second pumping syringe moves to expel the fluid from the barrel towards the patient.

Clause 38: The pump of clause 37, wherein the first and second inflow valves and the first and second outflow valves comprise one-way check valves.

Clause 39: The pump of clause 37 or clause 38, further comprising a valve set connected to the first pumping syringe, the second pumping syringe, and the reservoir, the valve set comprising: a housing enclosing at least one fluid chamber; an inflow port, wherein the fluid passes from the reservoir to the at least one fluid chamber through the inflow port; a first syringe port fluidly connected to an interior of the barrel of the first pumping syringe, wherein the fluid passes between the interior of the barrel of the first pumping syringe and the at least one fluid chamber through the first syringe port; a second syringe port fluidly connected to an interior of the barrel of the second pumping syringe, wherein the fluid passes between the interior of the barrel of the second pumping syringe and the at least one fluid chamber through the second syringe port; a first outflow port, wherein the fluid passes from the barrel of the first pumping syringe toward the patient through the first outflow port; and a second outflow port, wherein the fluid passes from the barrel of the second pumping syringe toward the patient through the second outflow port.

Clause 40: The pump of clause 39, wherein the first outflow valve and the second outflow valve are integral with the first outflow port and the second outflow port, respectively.

Clause 41: The pump of any of clauses 32-40, further comprising a pump controller electronically coupled to the first actuator and the second actuator, configured to cause the plunger of the first pumping syringe and the second pumping syringe to move substantially simultaneously in opposite directions.

Clause 42: The pump of clause 41, wherein the pump controller is configured to cause the first and second actuators to repeatedly move plungers back and forth through the barrels of the first and second pumping syringes.

Clause 43: The pump of clause 42, wherein repeated simultaneous movement of the plungers in opposite directions causes fluid to be expelled from the pump toward the patient as a continuous or substantially continuous flow.

Clause 44: The pump of any of clauses 41-43, wherein the reservoir is removably connected to other portions of the pump so that the reservoir can be removed and replaced while the pump controller continues to cause the first and second actuators to move the plungers through the barrels of the first and second pumping syringes.

Clause 45: A fluid infusion system, comprising: the pump of any of clauses 1-44; the reservoir fluidly connected to the pump containing the fluid to be delivered to the patient; and at least one patient line fluidly connected to the barrel of the at least one pumping syringe at a position distal to the at least one outflow valve, wherein the at least one patient line is configured to deliver the fluid from the pump to vasculature of the patient.

Clause 46: The fluid infusion system of clause 45, wherein the at least one pumping syringe comprises a first pumping syringe and a second pumping syringe, and wherein the at least one actuator comprises a first actuator that moves the plunger of the first pumping syringe and a second actuator that moves the plunger of the second pumping syringe.

Clause 47: The fluid infusion system of clause 46, wherein the at least one patient line comprises a first patient line configured to deliver fluid expelled from the first pumping syringe to the patient and a second patient line, separate from the first patient line, configured to deliver fluid expelled from the second pumping syringe to the patient.

Clause 48: The fluid infusion system of clause 46 or clause 47, wherein the at least one patient fluid line comprises a common patient line configured to deliver the fluid expelled from the first pumping syringe and from the second pumping syringe to the patient.

Clause 49: The fluid infusion system of clause 48, wherein the at least one patient line comprises a patient central line.

Clause 50: The fluid infusion system of any of clauses 45-49, wherein the reservoir comprising a reservoir syringe removably connected to the pump, wherein the reservoir syringe can be removed from the pump and replaced with another reservoir syringe while the at least one actuator continues to move the plunger through the barrel of the at least one pumping syringe.

Clause 51: The fluid infusion system of any of clauses 45-50, wherein the reservoir comprises an intravenous bag fluidly connected to the pump by at least one fluid conduit.

Clause 52: A method for fluid infusion to a patient, the method comprising: attaching the pump of any of clauses 1-44 to the reservoir, such that the reservoir is in fluid communication with the barrel of the at least one pumping syringe; placing the pump in fluid communication with vasculature of the patient; and causing the at least one actuator of the pump to repeatedly move the plunger through the barrel of the at least one pumping syringe, thereby causing the fluid to pass from the reservoir into the barrel of the at least one pumping syringe and from the barrel of the at least one pumping syringe to the patient.

Clause 53: The method of clause 52, wherein fluid passes from the pump toward the patient as a continuous, substantially continuous, semi-continuous, or intermittent flow.

Clause 54: The method of clause 52 or clause 53, wherein placing the pump in fluid communication with the vasculature of the patient comprises attaching the at least one outflow port of the pump to the at least one patient line extending from a blood vessel of the patient.

Clause 55: The method of any of clauses 52-54, wherein the at least one pumping syringe of the pump comprises a first pumping syringe and a second pumping syringe, and wherein placing the pump in fluid communication with the vasculature of the patient comprises attaching a first outlet of the pump, which is fluidly connected to the first pumping syringe, to a first patient line extending from the blood vessel of the patient and attaching a second outlet of the pump, which is fluidly connected to the second pumping syringe, to a second patient line extending from the blood vessel of the patient.

Clause 56: The method of any of clauses 52-55, wherein the at least one pumping syringe of the pump comprises the first pumping syringe and the second pumping syringe, and wherein placing the pump in fluid communication with the vasculature of the patient comprises attaching the first outlet of the pump, which is fluidly connected to the first pumping syringe, and the second outlet of the pump, which is fluidly connected to the second pumping syringe, to a common patient line extending from the blood vessel of the patient.

Clause 57: The method of any of clauses 52-56, wherein attaching the pump to the reservoir comprises attaching the pump to at least one of a syringe and an intravenous bag.

Clause 58: The method of any of clauses 52-57, further comprising removing the reservoir from the pump and replacing the reservoir with a new reservoir while the at least one actuator continues to move the plunger through the at least one pumping syringe.

### BRIEF DESCRIPTION OF THE DRAWINGS

FIG. 1A is a perspective view of a medical infusion pump, according to an aspect of the disclosure.

FIG. 1B is a cross-sectional view of the medical infusion pump of FIG. 1A.

FIG. 1C is a schematic drawing illustrating electrical components of the medical infusion pump of FIG. 1A.

FIG. 2A is a perspective view of another example of a medical infusion pump, according to an aspect of the present disclosure.

FIG. 2B is a side view of the medical infusion pump of FIG. 2A.

FIG. 2C is a cross-sectional view of the medical infusion pump of FIG. 2A.

FIG. 3A is a perspective view of an intravenous (IV) bag, as known in the prior art.

FIG. 3B is a schematic drawing of a fluid infusion system, according to an aspect of the present disclosure, including a medical infusion pump and an intravenous (IV) bag.

FIG. 4A is a perspective view of another example of a medical infusion pump, according to an aspect of the present disclosure.

FIG. 4B is a cross-sectional view of the medical infusion pump of FIG. 4A.

FIG. 4C is a schematic drawing illustrating electrical components of the medical infusion pump of FIG. 4A in a first configuration.

FIG. 4D is a schematic drawing illustrating electrical components of the medical infusion pump of FIG. 4A in a second configuration.

FIG. 5 is a flow chart illustrating a method for delivering a fluid to a patient with a medical infusion pump, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used herein, the singular form of “a”, “an”, and “the” include plural referents unless the context clearly dictates otherwise.

All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

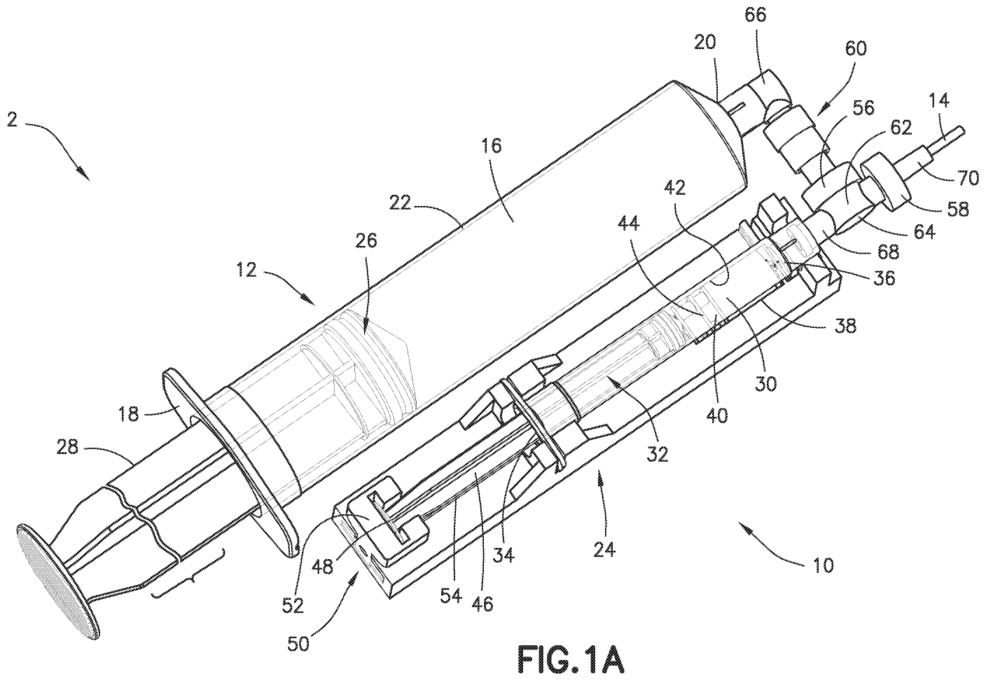

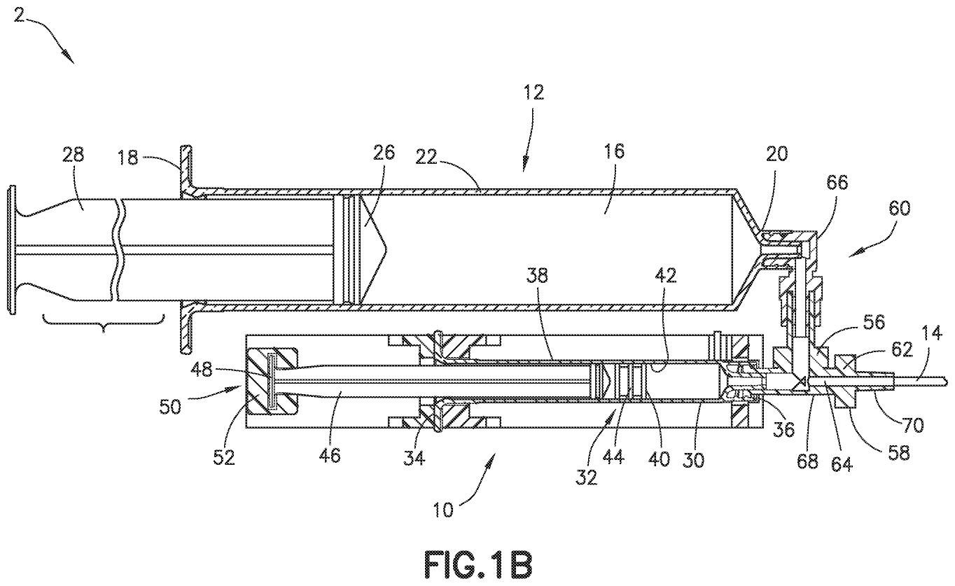

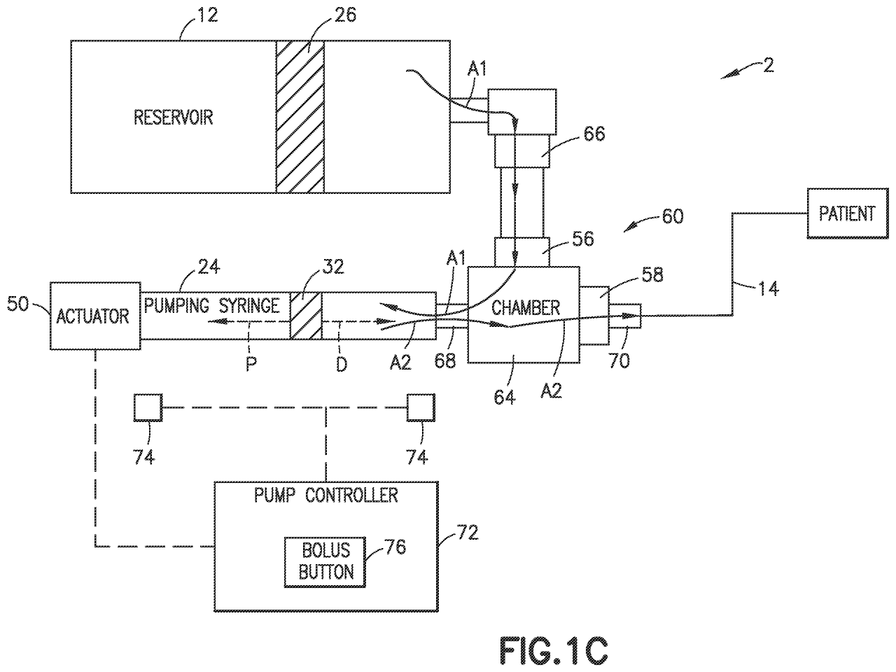

With reference to the figures, the present disclosure is directed to a wearable injection and/or infusion device, such as wearable medical infusion pumps 10, 110, configured to deliver a medical fluid from a fluid reservoir to a patient through one or more patient fluid lines. Examples of pumps 10, 110 for delivering the medical fluid to active (e.g., ambulatory) patients in hospital and in home settings are shown in FIGS. 1A-4D. The medical infusion pumps 10, 110 disclosed herein can be used for various medical procedures and applications, such as Keep Vein Open (KVO) and medication infusion applications.

The pumps 10, 110 of the present disclosure can be configured to provide a continuous, substantially continuous, semi-continuous, and/or intermittent flow of medical fluid to a patient. As used herein, a “continuous” flow can refer to a fluid flow having an unchanging flow rate and/or flow volume for a total duration of a fluid infusion procedure or event. A “substantially continuous flow” refers to a fluid flow having a flow rate and/or flow volume that changes by only a small amount (e.g., changes by less than about 10%) over the course of an infusion procedure or event. A “semi-continuous” flow can refer to a fluid flow that occurs continuously, but which is interrupted by consistent pauses (e.g., drops in fluid flow rate and/or flow volume) for a duration of an infusion procedure. For example, fluid may flow from the pump 10, 110 as a series of equally spaced pulses separated by durations when no fluid flows from the pump 10, 110. An “intermittent” fluid flow refers to a fluid flow that occurs at irregular intervals and which is not continuous or steady. For example, as described in further detail herein, the pump 10, 110 can include a bolus button, which causes the pump 10, 110 to initiate flow of the medical fluid to the patient. Fluid can be intermittently delivered to the patient only when the bolus button is pressed. At other times, no fluid may be delivered to the patient by the pump 10, 110.

The pumps 10, 110 of the present disclosure can be configured to provide improved accuracy and to be smaller in size compared to conventional medical infusion pumps. For example, the pumps 10, 110 of the present disclosure can include pumping syringes that are a small volume (e.g., about 3 mL to about 5 mL) and are controlled by mini-syringe actuators and flow control valves. Due to the small size of the pump components, the pumps 10, 110 can be configured for pumping doses of the medical fluid from a reservoir, such as an intravenous (IV) bag or a syringe, into a patient's central or intravenous (IV) line with tightly controlled tolerances for dose volume. The medical fluid can include any fluids commonly delivered to patients during medical procedures and/or to treat chronic or acute medical conditions, as are known in the art. For example, the pumps 10, 110 can be used for delivering medical fluids including fluids for flushing an IV line (e.g., saline solution), liquid therapeutic agents or medications, and/or a total parenteral nutrient (TPN) liquid. Also, as described in further detail herein, the small pumping syringes of the pumps 10, 110 do not constrain the medical fluid to be injected to the patient. Instead, the medical fluid is contained in a separate reservoir, such as the separate syringe or intravenous (IV) bag connected to the pump 10, 110. The medical fluid is drawn from the separate reservoir in small volumes and immediately provided to the patient. In some examples, the separate reservoir can be removed from the pump 10, 110 and replaced with a new reservoir. For example, the pumps 10, 110 can be configured to allow a user to remove an empty fluid reservoir from the pump 10, 110 and to attach a new full reservoir to the pump 10, 110. In some examples, reservoirs can be replaced while the pump 10, 110 continues to operate to provide the medical fluid to the patient. Accordingly, removing and replacing the fluid reservoir may not interrupt the flow of medical fluid to the patient.

Wearable Infusion Pump with Single Pumping Syringe

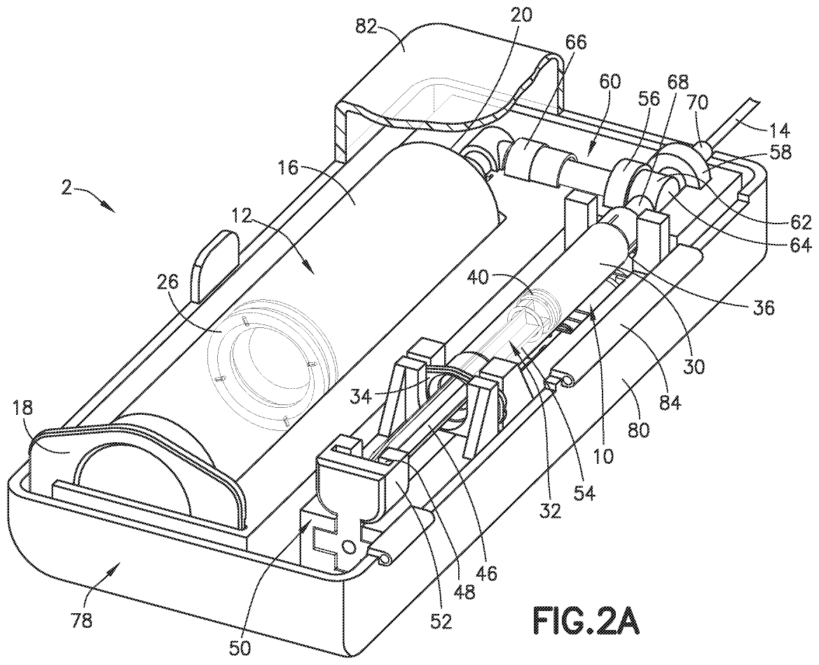

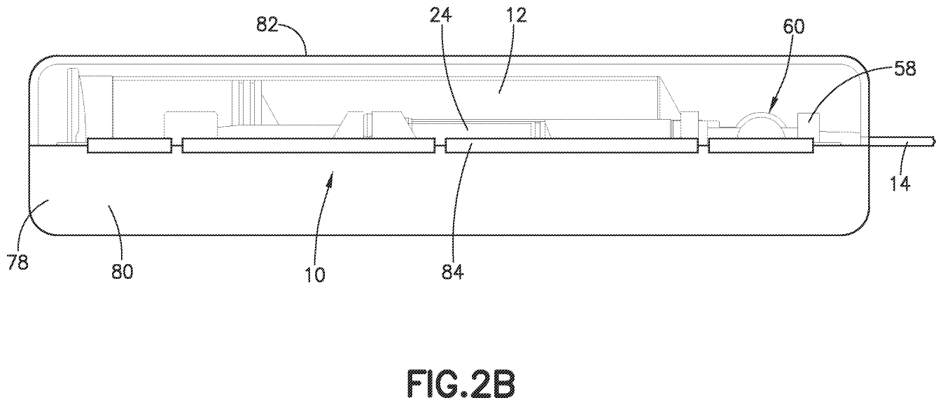

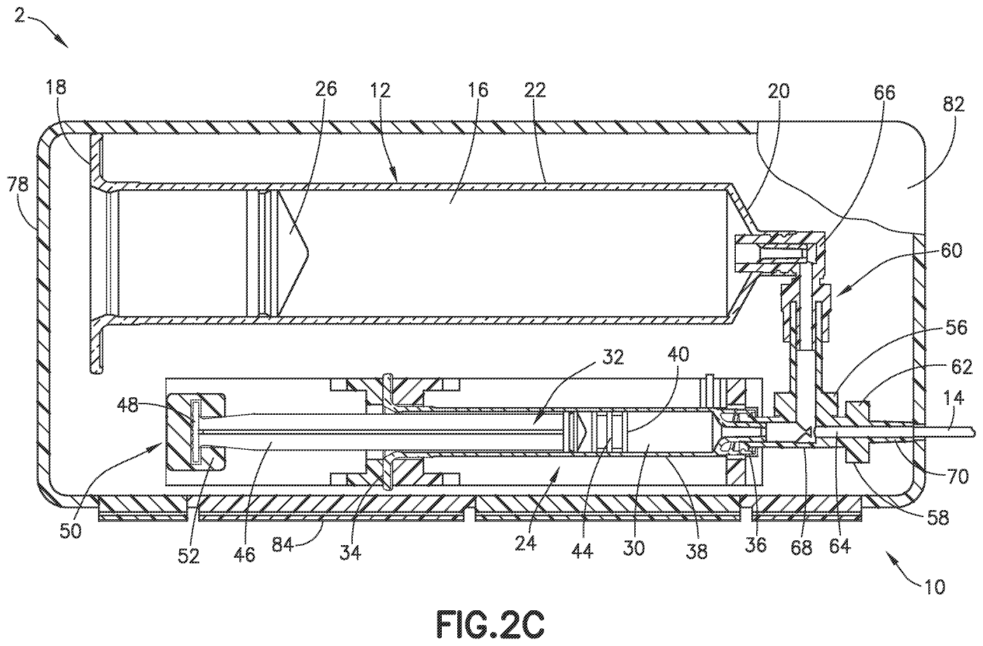

FIGS. 1A-2D illustrate an exemplary wearable medical infusion pump 10 for delivering, for example, a dose of a therapeutically effective amount of a medical fluid from a fluid containing reservoir, such as a reservoir syringe 12 or intravenous bag, to a patient at a predetermined delivery rate through a patient line 14, such as a vascular access device or intravenous catheter. The pump 10 can be used with a fluid infusion system 2, including the reservoir syringe 12 and the patient line 14 for delivering the medical fluid to the patient.

As used herein, a “wearable” medical device refers to a portable device that can be attached to and/or carried by the patient in a substantially hands free manner. For example, the wearable device can be connected to a percutaneous patient line or catheter and carried in a garment or support structure, such as a pocket, belt, holster, sling, backpack, fanny pack, or another wearable support structure, as are known in the art. In other examples, the wearable device can comprise an adhesive patch or sticker that can be adhered to the patient's skin. In some examples, the wearable device is a stand-alone device that is not connected to any other structures or devices, such as stationary support frames, external stationary power sources, or stationary fluid containers. The patient can be an ambulatory patient, meaning a patient that is capable of moving around (e.g., standing, walking, exercising, and/or performing other daily activities), while wearing the pump 10. For example, the wearable pump 10 can be configured for use in home-care settings or in a hospital for patients that are not confined to a bed and/or are capable of moving around independently.

As shown in FIGS. 1A-2D, the fluid reservoir can be a syringe (referred to herein as the reservoir syringe 12) having a barrel 16 comprising an open proximal end 18, a distal end 20 including a nozzle, fluid inlet/outlet, or fluid port that directly or indirectly connects to the pump 10, and a cylindrical sidewall 22 extending between the proximal end 18 and the distal end 20 of the barrel 16. The barrel 16 of the reservoir syringe 12 can have a large volume containing, for example, an entire dose of a medical fluid to be infused to a patient. For example, the barrel 16 can contain about 60 mL to about 100 mL or more of the medical fluid for infusion. The reservoir syringe 12 can also include a plunger 26 or stopper for containing the medical fluid in the reservoir barrel 16. In some examples, the reservoir syringe 12 can also include a plunger rod 28 extending proximally from the plunger 26. However, the plunger 26 or plunger rod 28 may not be needed because, as described in further detail herein, the pump 10 siphons the medical fluid through the nozzle or fluid port at the distal end 20 of the reservoir syringe 12, meaning that the user does not need to use the plunger rod 28 to manually advance the plunger 26 through the barrel 16 of the reservoir syringe 12.

As previously described, the medical fluid contained in the reservoir syringe 12 can include, for example, saline solution, a therapeutic agent, and/or TPN liquid, as well as any other medical fluid provided to a patient by infusion during a medical procedure and/or for treatment of chronic or acute conditions, as are known in the art. Exemplary therapeutic agents can include, for example, drugs, chemicals, biological, or biochemical substances that, when delivered in a therapeutically effective amount to the patient, achieve a desired therapeutic effect.

The infusion pump 10 further comprises a pumping syringe 24 including a barrel 30 and a plunger 32 or stopper movable through the barrel 30. The barrel 30 of the pumping syringe 24 can include an open proximal end 34, a distal end 36 including a nozzle or fluid port, and a cylindrical sidewall 38 extending between the proximal end 34 and the distal end 36 of the pumping syringe barrel 30. The barrel 30 contains a volume of fluid which is significantly smaller than the fluid volume contained by the reservoir syringe 12. For example, the pumping syringe 24 can contain about 3 mL to about 5 mL of fluid, while the reservoir syringe 12 contains about 60 mL to 100 mL or more of the medical fluid to be infused to the patient. Beneficially, the relatively small size of the pumping syringe 24 means that a total size of the pump 10 can be smaller than other infusion pumps known in the art, which often include a reservoir large enough to contain an entire dose of fluid to be delivered to the patient.

In some examples, the plunger 32 of the pumping syringe 24 includes an elastomeric stopper 40 that seals against an inner surface 42 of the cylindrical sidewall 38. The stopper 40 can be similar in structure to stoppers used in conventional syringes, as are known in the art. For example, as shown in FIGS. 1A and 1B, the stopper 40 can include a cylindrical body with one or more annular elastomeric ribs 44 extending around the body that form the seal with the inner surface 42 of the barrel 30. The plunger 32 can also include a plunger rod 46 connected to a proximal end of the stopper 40 for moving the stopper 40 through the barrel 30 to aspirate the medical fluid into the barrel 30 through the nozzle or fluid port at the distal end 36 of the barrel 30 and to expel the medical fluid from the barrel 30 through the nozzle or fluid port. In some examples, the plunger rod 46 includes a handle or grip 48 at a proximal end of the rod 46 similar to the thumb-press plate of a conventional syringe.

The medical infusion pump 10 also includes an actuator 50 for moving the plunger 32 through the barrel 30 of the pumping syringe 24 to draw the medical fluid from the reservoir syringe 12 into the barrel 30 and to expel the medical fluid from the barrel 30 towards the patient. The actuator 50 can be an electromechanical device including mechanical components for moving the plunger 32 through the barrel 30 and associated electrical circuitry for controlling movement of the plunger 32. For example, the actuator 50 can be a linear actuator that engages the plunger 32 to move the plunger 32 through the barrel 30. In some examples, the actuator 50 comprises a bracket or connector for engaging the plunger rod 46 and a drive device or assembly for advancing and retracting the bracket or connector. For example, the actuator 50 can include a retainer 52 sized to receive and/or engage a handle 48 or grip of the plunger rod 46. The actuator 50 can further include a track 54 positioned below the plunger rod 46. The actuator 50 can be configured to move the retainer 52 back and forth along the track 54, thereby moving the elastomeric stopper 40 through the barrel 30 of the pumping syringe 24.

With continued reference to FIGS. 1A-2D, the wearable infusion pump 10 further comprises valves for controlling the flow of the medical fluid from the reservoir syringe 12 to the pumping syringe 24 and from the pumping syringe 24 to the patient line 14. For example, the pump 10 can comprise an inflow valve 56 between the reservoir syringe 12 and the pumping syringe barrel 30 and an outflow valve 58 between the pumping syringe barrel 30 and the patient. The inflow valve 56 and the outflow valve 58 can be one-way check valves, as are known in the art. As used herein, a “check valve” means a valve that automatically opens or closes in response to changes in fluid flow and/or fluid pressure. For example, the inflow valve 56 can be configured to automatically open when the plunger 32 of the pumping syringe 24 moves in a proximal direction (shown by arrow P in FIG. 1C) to aspirate the fluid from the reservoir syringe 12 into the pumping syringe barrel 30 in a direction of arrow A1 (shown in FIG. 1C). The outflow valve 58 can be positioned to automatically close when the plunger 32 moves through the barrel 30 of the pumping syringe 24 in the proximal direction. The outflow valve 58 is configured to automatically open when the plunger 32 moves in a distal direction (shown by arrow D in FIG. 1C) to expel the medical fluid from the barrel 30 of the pumping syringe 24. The expelled fluid moves from the barrel 30 of the pumping syringe 24, through the open outflow valve 58, and towards the patient line 14 and patient, as shown by arrow A2 (shown in FIG. 1C). In addition, moving the plunger 32 in the distal direction (shown by arrow D) causes the inflow valve 56 to automatically close so that fluid expelled from the barrel 30 of the pumping syringe 24 does not return to the reservoir syringe 12.

In some examples, the valves 56, 58 are joined together in a valve set 60 connected to the reservoir syringe 12, the pumping syringe 24, and/or the patient line 14. The valve set 60 can include, for example, a housing 62 enclosing a fluid chamber 64. The housing 62 can be a tubular enclosure, fluid conduit, segment of pipe, or other structures containing or removably connected to the inflow valve 56 and the outflow valve 58. In some examples, the valve set 60 comprises an inflow port 66, a syringe port 68, and an outflow port 70. The ports 66, 68, 70 can be integral with or removably connected to the housing 62. The inflow port 66 can be positioned to allow the medical fluid to pass from the reservoir syringe 12 to the fluid chamber 64 of the valve set 60 through the inflow port 66. The syringe port 68 can be fluidly connected to an interior of the barrel 30 of the pumping syringe 24 and arranged to permit the medical fluid to pass between the interior of the barrel 30 and the fluid chamber 64 through the syringe port 68. The outflow port 70 can be positioned to permit the medical fluid to pass from the fluid chamber 64 to the patient through the outflow port 70.

The ports 66, 68, 70 can include various connecting structures, such as luer connectors, screw threads, and/or press-fit connectors, as are known in the art, for attaching the valve set 60 to the reservoir syringe 12, the pumping syringe 24, and the patient line 14. For example, the inflow port 66 and/or syringe port 68 can be a female luer connector sized to receive the nozzle or fluid port at the distal end 20, 36 of the barrel 16, 30 of the reservoir syringe 12 or pumping syringe 24. The outflow port 70 can include a connector, such as a luer connector, sized to engage a proximal end of the patient line 14 or intravenous catheter for attaching the pump 10 to the patient line 14 or catheter. In some examples, the inflow valve 56 is integral with the inflow port 66 of the valve set 60. In other examples, the inflow valve 56 can be positioned in the valve set 60, such that fluid passes through the inflow port 66 to the inflow valve 56 through fluid conduits of the valve set 60. In other examples, the inflow valve 56 can be a separate structure that removably connects to the inflow port 66 so that medical fluid from the reservoir syringe 12 passes through the inflow valve 56 before passing through the inflow port 66 into the valve set 60. Similarly, the outflow valve 58 can be integral with the outflow port 70. Alternatively, the outflow valve 58 can be positioned in the valve set 60, such that fluid passes from the fluid chamber 64, past the outflow valve 58, and through the outflow port 70. In other examples, the outflow valve 58 can be a separate structure that attaches directly or indirectly to the outflow port 70, such that fluid passes from the fluid chamber 64, through the outflow port 70, and then through the outflow valve 58.

With specific reference to FIG. 1C, in some examples, the pump 10 further comprises a pump controller 72 electrically coupled to the actuator 50 for controlling operation of the actuator 50. The pump controller 72 can include one or more computer processors and associated computer-readable memory including instructions for operating the actuator 50. For example, the memory can include operating parameters that cause the actuator 50 to move the plunger 32 through the barrel 30 at a predetermined plunger speed to achieve a target flow rate and/or flow volume. The memory can also include information about preferred operating routines for the pump 10. For example, for some infusions or therapies, the pump controller 72 can be configured to provide medical fluid to the patient at a first flow rate for a period of time and/or until a predetermined first volume of the medical fluid is delivered to the patient followed by providing the fluid to the patient at a different second flow rate for another period of time or until a predetermined second volume of the medical fluid is delivered to the patient.

The pump controller 72 is also configured to monitor or detect movement of the plunger 32 through the barrel 30 of the pumping syringe 24 in order to determine when to change direction of the plunger 32. In this way, the pump controller 72 can control back and forth movement of the plunger 32 through the barrel 30, so that the pump 10 provides a continuous, substantially continuous, or semi-continuous flow of medical fluid to the patient. In some examples, the pump controller 72 can be configured to monitor a torque exerted by the actuator 50 on the plunger 32 to detect when the plunger 32 approaches an end-of-stroke position at the proximal end 34 or distal end 36 of the barrel 30. For example, torque can increase as the plunger 32 approaches the proximal end 34 and/or the distal end 36 of the barrel 30. When the detected torque increases above a threshold value, the pump controller 72 can cause the actuator 50 to change direction of the plunger 32. For example, a torque exerted by the actuator 50 increases as the actuator 50 moves the plunger 32 in a distal direction to expel fluid from the pumping syringe 24 toward the patient. As the plunger 32 approaches the distal end 36 of the pumping syringe 24 near to the syringe nozzle or fluid port, the measured torque increases above the target torque value. When the measured torque is greater that the target value, the pump controller 72 causes the actuator 50 to change direction and to begin moving the plunger 32 in the proximal direction to aspirate fluid from the reservoir syringe 12 into the barrel 30 of the pumping syringe 24.

In some examples, the pump 10 can also include sensors 74 (shown in FIG. 1C) in electrical communication with the pump controller 72 for detecting a position of the plunger 32 within the barrel 30 of the pumping syringe 24. For example, one or more position sensors 74 can be positioned proximate to the barrel 30 to detect when the plunger 32 approaches the proximal end 34 or the distal end 36 of the pumping syringe barrel 30. The sensors 74 can be optical sensors that detect images of the plunger 32 and/or plunger rod 46 to determine the plunger's 32 position. In other examples, the sensors 74 can be distance sensors, such as a laser range finder, that detect, for example, a distance between the plunger 32 and the proximal end 34 of the barrel 30. In use, when the plunger 32 reaches the end-of-stroke position at the proximal end 34 or the distal end 36 of the barrel 30, as detected by the sensors 74, the pump controller 72 can cause the actuator 50 to change the movement direction of the plunger 32 so that the plunger 32 continues to repeatedly move back and forth through the pumping syringe barrel 30.

In some examples, the pump 10 can include inputs, such as buttons or a touchscreen display, allowing a user, such as a caregiver or the patient, to adjust or control operation of the pump 10. For example, the user can manipulate the inputs causing the pump controller 72 to adjust operating parameters of the pump 10, such as by starting an infusion procedure, pausing an infusion procedure, stopping an infusion procedure, and/or adjusting a flow rate and/or flow volume for the medical fluid being delivered from the pump 10 to the patient. For example, the pump controller 72 can be configured to change a rate at which the actuator 50 moves the plunger 32 through the barrel 30 of the pumping syringe 24 based on information entered by the caregiver or patient. In one example, an input button can be a bolus input button 76 electrically coupled to the pump controller 72 that can be pressed by the user (e.g., the caregiver or patient) to cause the pump 10 to deliver a bolus of the medical fluid from the pump 10 to the patient. The pump controller 72 can be configured to receive an indication when the bolus input button 76 is pressed by a user and can automatically cause the actuator 50 to increase a rate at which the plunger 32 moves through the barrel 30 for a predetermined duration to provide a medical fluid bolus (e.g., a short duration surge of medical fluid) to the patient.

With reference to FIGS. 2A-2D, in some examples, the pump 10 can be entirely or partially contained within an enclosure 78 or housing for protecting components of the pump 10 and/or for making the pump 10 more convenient to wear or carry. For example, the pumping syringe 24 and valve set 60 could be contained within the enclosure 78. In some examples, the entire reservoir syringe 12 can also be contained in the enclosure 78, while, in other examples, a portion of the reservoir syringe 12 may be outside of the enclosure 78 so that the reservoir syringe 12 can be easily removed from the pump 10 and replaced when empty. The enclosure 78 or housing can be formed by a rigid protective material, such as hard plastic. For example, the enclosure 78 can be formed from high density polyethylene or polycarbonate materials. In other examples, the enclosure 78 can be made from glass, metal, ceramic materials, or combinations thereof. The enclosure 78 can include padding or insulation for protecting the reservoir syringe 12 and other components of the pump 10 from damage if, for example, the pump 10 is dropped, shaken, or otherwise mistreated.

In some examples, the wearable pump 10 can be used for delivering therapeutic agents to patients that are controlled substances, such as opioids, stimulants, depressants, hallucinogens, or anabolic steroids. For such controlled substances, the pump 10 can be a controlled-access device that prevents the patient from accessing the reservoir syringe 12 or from obtaining more than a prescribed dose of the medical fluid at one time. In order to make the pump 10 controlled access, all components of the pump 10, including the entire reservoir syringe 12, can be entirely contained within the enclosure 78 or housing so that the patient cannot remove the reservoir syringe 12 from the pump 10 to access the medical fluid contained therein. For the controlled substances, it may also be important to ensure that the therapeutic agent is only released from the pump 10 at intended times and/or at desired flow rates. In particular, it may be important to ensure that the therapeutic agent cannot be siphoned off or drained from the pump 10 allowing the patient to obtain a large volume of the medical fluid at one time. In order to prevent patients from attempting to obtain large volumes of the controlled-access solution or medical fluid at one time, a cracking pressure of the inflow valve 56 and/or the outflow valve 58 can be made to be greater than atmospheric pressure. As used herein, the “cracking pressure” of a check valve refers to a minimum upstream pressure required to open the check valve enough to allow a detectable flow to pass through the valve. Making the cracking pressure of the inflow valve 56 and/or the outflow valve 58 greater than atmospheric pressure ensures that the valves 56, 58 cannot be forced open by, for example, providing a negative pressure at the outflow valve 58 sufficient to draw fluid from the syringe reservoir 12 through the valves 56, 58.

The enclosure 78 can be a variety of shapes and configurations depending, for example, upon a size and shape of components of the pump 10 and a degree of safety or security desired for the components of the pump 10. For example, as shown in FIGS. 2A-2D, the enclosure 78 is a two-piece clam shell enclosure comprising a base 80 including cavities or compartments sized to receive the reservoir syringe 12, pumping syringe 24, and valve set 60. A top 82 is connected to the base 80 along a hinge 84 for opening and closing the top 82. In some examples, the patient may be able to open the pump 10 to remove an empty reservoir syringe 12 and to replace the empty reservoir syringe 12 with a full reservoir syringe 12. In that case, as shown in FIGS. 2A-2D, the top 82 can be formed from a transparent or translucent material so that the patient can easily see when the reservoir syringe 12 is empty and should be replaced.

In other examples, particularly for a controlled access device (e.g., a pump 10 containing a controlled substance, such as an opioid or another narcotic), the base 80 and the top 82 can be sealed or locked together preventing the patient from opening the pump 10 to access the reservoir syringe 12 contained therein. In that case, a clinician, such as a prescribing physician or another medical practitioner, may have a key or another tool for unlocking the pump 10 to replace an empty reservoir syringe 12. In other examples, the pump 10 can be a single-use device and can be discarded when the reservoir syringe 12 is empty. In that case, the enclosure 78 can be permanently closed during manufacture, preventing any individual from accessing the reservoir syringe 12 or other components of the pump 10 to obtain a controlled substance contained in the reservoir syringe 12.

Fluid Infusion System with Intravenous Bag and Infusion Pump

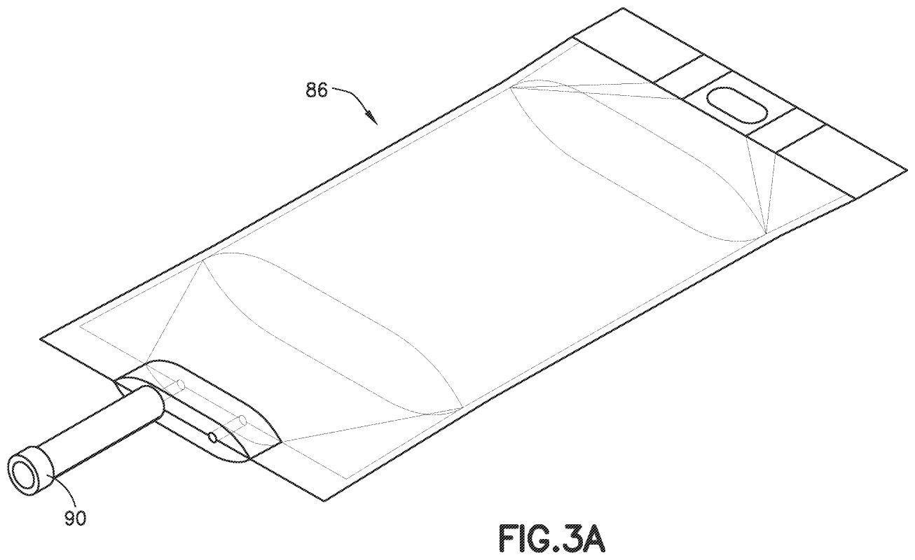

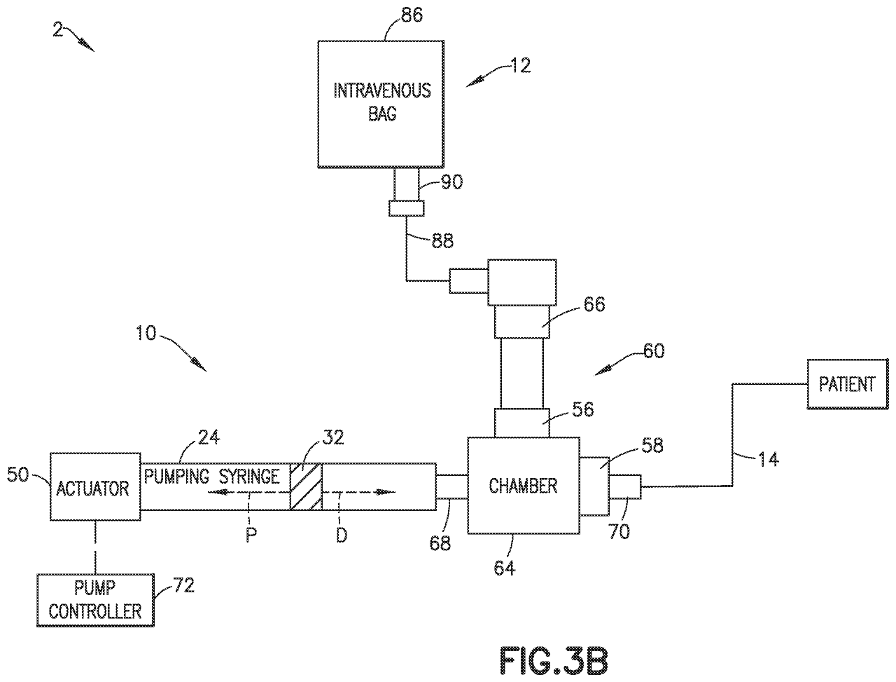

With reference to FIGS. 3A and 3B, in some examples, as previously described, the reservoir syringe 12 for the pump 10 can be an intravenous (IV) bag 86. The intravenous (IV) bag 86 can be an inflatable bag formed from one or more layers of a polymer material, such as a multilayer polyolefin film. In other examples, the IV bag 86 can be made from other flexible plastic materials, such as polyethylene, natural rubber, or latex. The IV bag 86 can include a sealed or sealable port 90 that can be directly or indirectly connected to the pump 10. For example, the port 90 can comprise a self-sealing septum configured to be pierced by a needle cannula or another fluid conduit for establishing fluid communication between the IV bag 86 and the pump 10. In some examples, the IV bag 86 can be wearable attached, for example, to the patient's upper arm and fluidly connected to the pump 10 by flexible tubing. An exemplary infusion system including a wearable IV bag, which can be used with the pump 10 of the present disclosure is described, for example, in U.S. Patent Appl. Pub. No. 2021/0085859, entitled “Medical Infusion Pump for Delivery of a Fluid”, which is incorporated herein by reference. In other examples, the IV bag 86 can be connected to a stand or another portable or stationary support, as are known in the art.

An exemplary intravenous (IV) bag 86 that can be used with the pumps 10 and fluid infusion systems 2 disclosed herein is shown in FIG. 3A. A fluid infusion system 2 for delivering a medical fluid from the IV bag 86 to the patient including the infusion pump 10 is shown in FIG. 3B. As shown in FIG. 3B, the IV bag 86 is connected to the inflow port 66 of pump 10 by a conduit 88, such as a segment of flexible medical tubing, that extends from the inflow port 66 to the port 90 of the IV bag 86. As in previous examples, the patient line 14 extends from the outflow port 70 of the pump 10 to the patient. As in previous examples, the medical fluid flows from the reservoir (the IV bag 86) to the pump 10 when the plunger 32 of the pumping syringe 24 moves in a proximal direction to draw the medical fluid into the barrel 30 of the pumping syringe 24. The medical fluid is expelled from the barrel 30 of the pumping syringe 24 as the plunger 32 moves in the distal direction.

Medical Infusion Pump with Dual Pumping Syringes

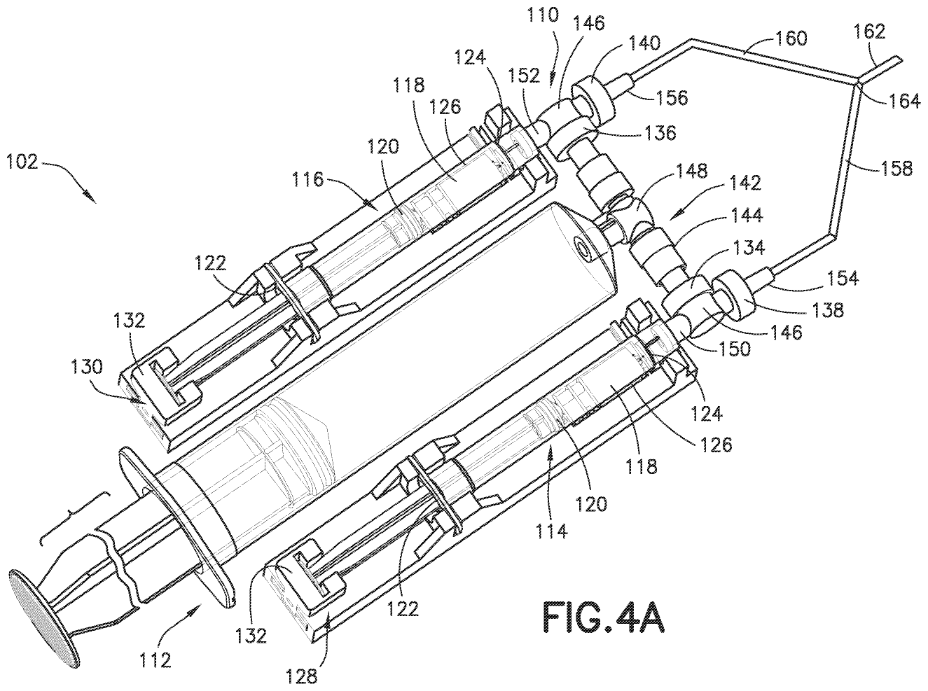

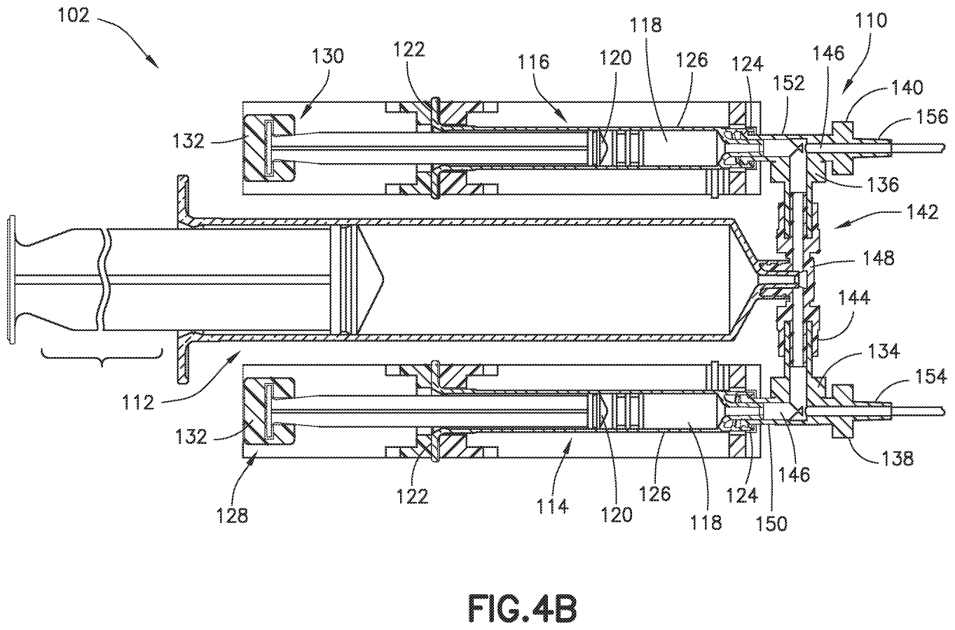

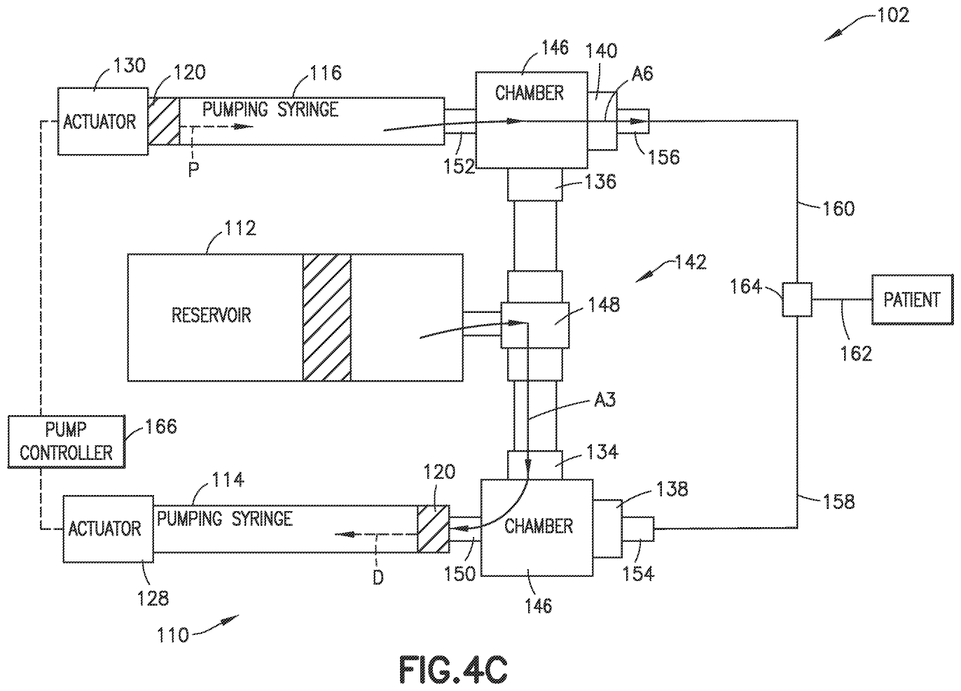

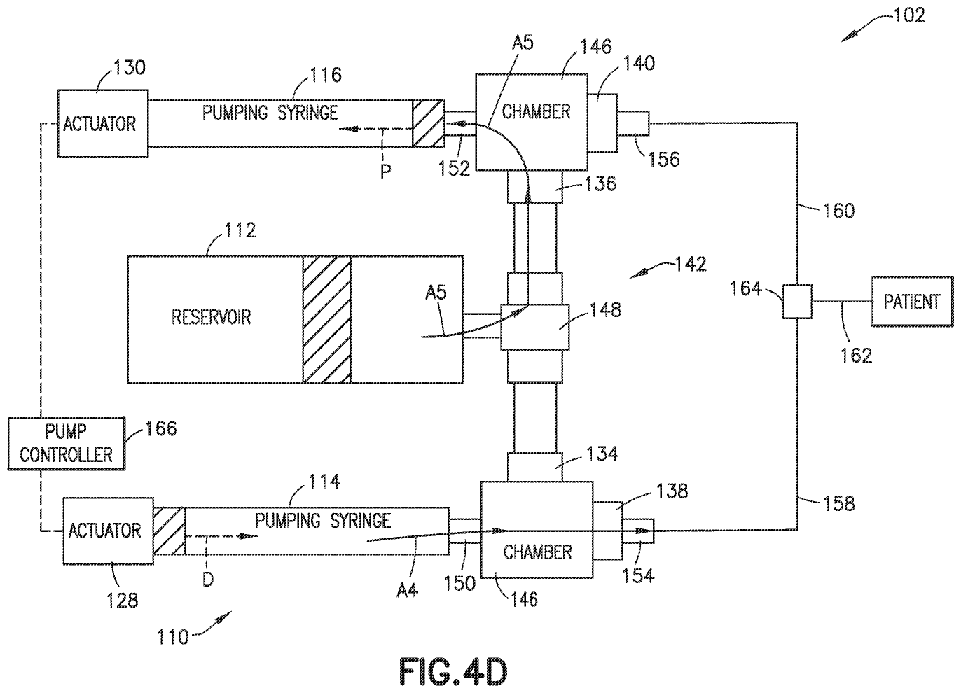

FIGS. 4A-4D illustrate a fluid infusion system 102 comprising a wearable medical infusion pump 110 for delivering medical fluid from a fluid reservoir, such as a reservoir syringe 112, to a patient with dual pumping syringes 114, 116. As in previous examples, the reservoir can be a reservoir syringe 112 (as shown in FIGS. 4A-4C), an IV bag, or other containers for holding medical fluid, as are known in the art. The pump 110 of FIGS. 4A-4C is not shown with an enclosure, casing, or housing. However, it is understood that, within the scope of the present disclosure, some or all of the components of the pump 110 can be provided in a housing or enclosure, such as the clam-shell enclosure illustrated in FIGS. 2A-2D, to protect components of the pump 110 and to ensure that the medical fluid in the reservoir syringe 112 is not tampered with or improperly drained from the pump 110.

In some examples, the pump 110 comprises a first pumping syringe 114 and a second pumping syringe 116. As in previous examples, each pumping syringe 114, 116 comprises a barrel 118 and a plunger 120 or stopper movable through the barrel 118. The barrel 118 can comprise an open proximal end 122, a distal end 124 comprising a nozzle or fluid port, and a cylindrical sidewall 126 extending therebetween. The pump 110 further comprises actuators for moving the plungers 120 of the first pumping syringe 114 and the second pumping syringe 116 through the barrels 118 to draw the medical fluid from the reservoir syringe 112 into the barrels 118, and to expel the medical fluid from the barrels 118 towards the patient. For example, the pump 110 can include a first actuator 128 for moving the plunger 120 of the first pumping syringe 114 and a second actuator 130 for moving the plunger 120 of the second pumping syringe 116. As in previous examples, the actuators 128, 130 can be electromechanical devices comprising a retainer 132 for engaging the plunger 120 of the pumping syringes 114, 116 and electrical circuitry for causing the retainers 132 to move the plungers 120 in proximal and distal directions through the barrels 118. As described in further detail hereinafter, the actuators 128, 130 can be configured to move the plunger 120 of the first pumping syringe 114 and the plunger of the second pumping syringe 116 simultaneously, but in opposite directions, which causes a continuous or substantially continuous fluid flow to be expelled from the pump 110 towards the patient.

The pump 110 further comprises inflow valves and outflow valves for directing fluid flow through the pump 110. As in previous examples, the inflow valves and the outflow valves can be one-way check valves. Also, the inflow valves and the outflow valves can be part of a valve set that can be removably connected to the pumping syringes 114, 116 and/or to the reservoir syringe 112. As shown in FIGS. 4A-4D, the inflow valves are positioned between the reservoir syringe 112 and the barrels 118 of the pumping syringes 114, 116. For example, the pump 110 can include a first inflow valve 134 between the reservoir syringe 112 and the barrel 118 of the first pumping syringe 114 that opens when the plunger 120 of the first pumping syringe 114 moves in the proximal direction (shown by arrow P in FIG. 4C) to aspirate the medical fluid from the reservoir syringe 112 into the barrel 118 of the first pumping syringe 114. Specifically, the aspirated fluid flows as shown by arrow A3 (in FIG. 4C). The first inflow valve 134 closes when the plunger 120 of the first pumping syringe 114 moves in the distal direction (shown by arrow D in FIG. 4D) to expel fluid from the barrel 118 of the first pumping syringe 114.

The pump 110 can also include a second inflow valve 136 between the reservoir syringe 112 and the barrel 118 of the second pumping syringe 116 that opens when the plunger 120 of the second pumping syringe 116 moves in the proximal direction (shown by arrow P in FIG. 4D) to aspirate the fluid from the reservoir syringe 112 into the barrel 118 of the second pumping syringe 116. Flow of fluid from the reservoir syringe 112 to the barrel 118 of the second pumping syringe 116 is shown by arrow A5 (in FIG. 4D). The second inflow valve 136 is configured to close when the plunger 120 of the second pumping syringe 116 moves to expel the medical fluid from the barrel 118 of the second pumping syringe 116 toward the patient in a direction of arrow A6 (in FIG. 4D).

The pump 110 further comprises outflow valves between the barrels 118 of the pumping syringes 114, 116 and the patient. For example, the pump 110 can include a first outflow valve 138 between the barrel 118 of the first pumping syringe 114 and the patient that opens when the plunger 120 of the first pumping syringe 114 moves to expel the medical fluid from the barrel 118 towards the patient, as shown in FIG. 4C. The first outflow valve 138 is closed when the plunger 120 of the first pumping syringe 114 moves in the proximal direction to aspirate fluid into the barrel 118 of the first pumping syringe 114.

The pump 110 can also include a second outflow valve 140 between the barrel 118 of the second pumping syringe 116 and the patient that opens when the plunger 120 of the second pumping syringe 116 moves to expel the medical fluid from the barrel 118 towards the patient, as shown in FIG. 4D. The second outflow valve 140 closes when the plunger 120 of the second pumping syringe 116 moves in the proximal direction to aspirate fluid into the barrel 118 of the second pumping syringe 116.

In some examples, the inflow valves 134, 136 and the outflow valves 138, 140 are components of a valve set 142 connected to the pumping syringes 114, 116 and the reservoir syringe 112. As in previous examples, the valve set 142 for the dual-syringe pump 110 can comprise a housing 144 enclosing fluid chambers 146 and an inflow port 148 positioned to allow fluid from the reservoir syringe 112 to pass through the inflow port 148 to the fluid chamber 146. The valve set 142 also includes a first syringe port 150 fluidly connected to an interior of the barrel 118 of the first pumping syringe 114 and a second syringe port 152 fluidly connected to an interior of the barrel 118 of the second pumping syringe 116. The medical fluid passes between the interior of the barrel 118 of the first pumping syringe 114 and the fluid chamber 146 through the first syringe port 150. In a similar manner, the medical fluid passes between the interior of the barrel 118 of the second pumping syringe 116 and the fluid chamber 146 through the second syringe port 152. The valve set 142 also includes a first outflow port 154 and a second outflow port 156. The medical fluid passes from the barrel 118 of the first pumping syringe 114 toward the patient through the first outflow port 154. In a similar manner, the fluid passes from the barrel 118 of the second pumping syringe 116 toward the patient through the second outflow port 156. In some examples, the outflow valves 138, 140 are integral with the outflow ports 154, 156. In other examples, the outflow valves 138, 140 and the outflow ports 154, 156 can be separate structures that are directly connected together or indirectly connected by one or more tubing segments.

In some examples, the medical fluid is expelled from the pump 110 through the outflow ports 154, 156 to one or more patient fluid lines, such as intravenous catheters, as are known in the art. For example, there can be a first patient fluid line 158 connected to the first outflow port 154 and a second patient fluid line 160 connected to the second outflow port 156. In some examples, the patient lines 158, 160 can be entirely separate such that fluid expelled from the first outflow port 154 passes through the first patient fluid line 158 to the patient's vasculature at a first location and fluid expelled from the second outflow port 156 passes through the second patient fluid line 160 to the patient's vasculature at a second location different from the first location. Including two separate patient lines 158, 160 means that fluid continues to be delivered to the patient even if one of the outflow ports 154, 156 or patient lines 158, 160 becomes occluded. Fluid flow through the separate patient lines 158, 160 is semi-continuous meaning that fluid passes through the first patient fluid line 158 when the plunger 120 of the first pumping syringe 114 moves in the distal direction (shown by arrow D in FIG. 4C) and the medical fluid passes through the second patient fluid line 160 when the plunger 120 of the second pumping syringe 116 moves in the distal direction (shown by the arrow D in FIG. 4D).

In other examples, patient fluid lines 158, 160 extending from the first outflow port 154 and the second outflow port 156 can join together prior to reaching the patient. For example, the first patient fluid line 158 and the second patient fluid line 160 may form a common patient line 162 extending from an intersection point 164 to vasculature of the patient. Fluid flow through the common patient line 162 can be a continuous or substantially continuous flow, meaning that the fluid flow continues both when the plunger 120 of the first pumping syringe 114 moves in the distal direction and when the plunger 120 of the second pumping syringe 116 moves in the distal direction.

With specific reference to FIGS. 4C and 4D, as in previous examples, the pump 110 can further comprise a pump controller 166. The pump controller 166 can be a computer device and/or computer processor(s) electronically coupled to the first actuator 128 and the second actuator 130. The pump controller 166 can be configured to cause the plunger 120 of the first pumping syringe 114 and the plunger 120 of the second pumping syringe 116 to move substantially simultaneously in opposite directions. For example, the pump controller 166 can be configured to cause the first actuator 128 to move the plunger 120 in the proximal direction (shown by arrow P in FIG. 4C) to draw the medical fluid into the barrel 118 of the first pumping syringe 114 while, simultaneously, causing the second actuator 130 to move the plunger 120 of the second pumping syringe 116 in the distal direction (shown by arrow D in FIG. 4C) to expel any medical fluid from the barrel 118 of the second pumping syringe 116 toward the patient. Once the plungers 120 reach their end-of-stroke positions (e.g., the proximal-most position for the plunger 120 of the first pumping syringe 114 and the distal-most position for the plunger 120 of the second pumping syringe 116), the actuators 128, 130 cause the plungers 120 to reverse direction, meaning that the medical fluid is expelled from the barrel 118 of the first pumping syringe 114 and the medical fluid is drawn into the barrel 118 of the second pumping syringe 116, as shown in FIG. 4D.

As in previous examples, the pump controller 166 can cause the actuators 128, 130 to move the plungers 120 back and forth through the syringe barrels 118 continuously for an entire infusion procedure or event. Moving the plungers 120 continuously through the syringe barrels 118, but in opposite directions, causes fluid to be expelled from the pump 110 toward the patient as a continuous or substantially continuous flow. As in previous examples, the reservoir syringe 112 can be removably connected to other portions of the pump 110 so that the reservoir 112 can be removed and replaced while the pump controller 166 continues to cause the first and second actuators 128, 130 to move the plungers 120 through the barrels 118 of the first and second pumping syringes 114, 116. Accordingly, an empty reservoir syringe 112 can be replaced with a full reservoir syringe 112 without interrupting operation of the pump controller 166 and/or actuators 128, 130 or the continuous or substantially continuous flow of the medical fluid to the patient.

### Fluid Infusion Methods

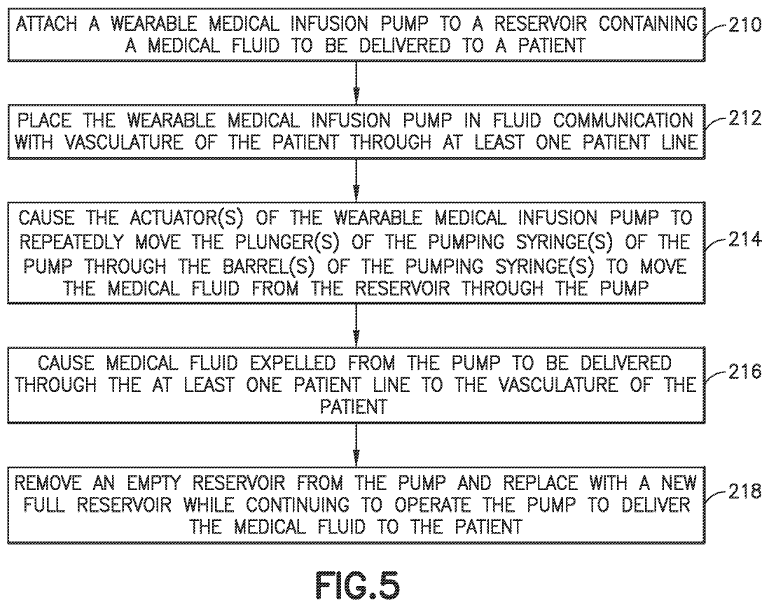

The pumps 10, 110 of the present disclosure are used for infusing a medical fluid to a patient through, for example, the patient line(s) 14, 158, 160 and/or intravenous catheter(s). With reference to FIG. 5, the method comprises a step 210 of attaching a wearable medical infusion pump, such as an infusion pump 10 comprising the single pumping syringe 24 (shown in FIGS. 1A-3B) or the dual pumping syringes 114, 116 (shown in FIGS. 4A-4D), of the present disclosure, to a reservoir. As previously described, the reservoir can be a reservoir syringe 12, 112, as shown in FIGS. 1A-2D and 4A-4C. In other examples, the reservoir can be an IV bag 86 (shown in FIGS. 3A and 3B) or any other convenient container for containing a medical fluid, such as a vial, canister, cartridge, or other containers for medical fluids, as are known in the art. Attaching the reservoir to the pump 10, 110 places an interior of the reservoir in fluid communication with an interior of the barrel 16, 118 of the pumping syringe(s) 24, 114, 116.

At step 212, the method further comprises placing the pump 10, 110 in fluid communication with vasculature of the patient. For example, placing the pump 10 in fluid communication with the vasculature of the patient can include attaching the outflow port 70 of the pump 10 to the patient line 14 fluidly connected to a blood vessel of the patient. The patient line 14 can be an intravenous catheter or similar infusion tool, as are known in the art. For a dual-syringe pump 110, placing the pump 110 in fluid communication with the vasculature of the patient includes attaching a first patient line 158 to a first outflow port 154 and attaching a second patient line 160 to a second outflow port 156 of the pump 110. As previously described, in some examples the patient lines 158, 160 are separate along their entire lengths. In other examples, the patient lines 158, 160 can be connected together to form the common patient line 162. After attaching the outflow port(s) 70, 154, 156 of the pump 10, 110 to the patient line(s) 14, 158, 160, the pump 10, 110 can be secured to the patient. For example, as previously described, the pump 10, 110 can be a wearable pump that remains external to the patient's body. In some examples, the wearable pump 10, 110 can be adhered to the patient's skin by adhesive patches or members, such as stickers or tape. In other examples, the pump 10, 110 can be placed in a wearable carrier or support structure, such as a pocket, belt, vest, fanny pack, or backpack.

At step 214, the method further comprises causing the actuator(s) 50, 128, 130 of the pump 10, 110 to repeatedly move the plunger(s) 32, 120 through the barrel(s) 30, 118 of the pumping syringe(s) 24, 114, 116, thereby causing the medical fluid to travel from the reservoir, such as the reservoir syringe 12, 112, into the barrel(s) 30, 118 of the pumping syringe(s) 24, 114, 116, and from the barrel(s) 30, 118 of the pumping syringe(s) 24, 114, 116 to the patient. For example, for a pump 10 with a single pumping syringe 24 (shown in FIGS. 1A-2D), when the actuator 50 moves the plunger 32 through the barrel 30 of the pumping syringe 24 in a proximal direction (e.g., towards the proximal end 34 of the pumping syringe barrel 30), the inflow valve 56 opens and the medical fluid is drawn from the reservoir syringe 12 through the inflow valve 56 in a direction of arrow A1 (shown in FIG. 1C) to the interior of the barrel 30 of the pumping syringe 24. When the actuator 50 moves the plunger 32 in the distal direction towards the distal end 36 of the pumping syringe barrel 30, the inflow valve 56 closes and the outflow valve 58 opens. When the outflow valve 58 opens, the medical fluid is expelled from the pumping syringe barrel 30 and moves through the outflow valve 58 and to the patient line 14 extending from the pump 10.

For a pump 110 with dual pumping syringes (shown in FIGS. 4A-4D), the first and second actuators 128, 130 move the plungers 120 of the first pumping syringe 114 and the second pumping syringe 116 simultaneously, but in opposite directions. For example, the first actuator 128 can cause the plunger 120 of the first pumping syringe 114 to move in the proximal direction, shown by arrow P (in FIG. 4C), while the second actuator 130 moves the plunger 120 of the second pumping syringe 116 in the distal direction, shown by arrow D (in FIG. 4C). In that case, the first inflow valve 134 opens and the first outflow valve 138 closes, which prevents fluid from passing from the pump 110 towards the patient. Medical fluid is drawn from the reservoir syringe 112 through the first inflow valve 134 to the barrel 118 of the first pumping syringe 114 in a direction of arrow A3 (in FIG. 4C). The second inflow valve 136 is closed preventing the medical fluid from flowing to the barrel 118 of the second pumping syringe 116.

Once the plungers 120 reach end-of-stroke positions (e.g., the plunger 120 of the first pumping syringe 114 reaches its proximal-most position and the plunger 120 of the second pumping syringe 116 reaches its distal-most position), the direction of movement of the plungers 120 is reversed. Specifically, the first actuator 128 causes the plunger 120 of the first pumping syringe 114 to move in the distal direction (shown by arrow D in FIG. 4D) and the second actuator 130 causes the plunger 120 of the second pumping syringe 116 to move in a proximal direction as shown by arrow P (shown in FIG. 4D). When the plungers 120 move in these directions, the first inflow valve 134 closes and the first outflow valve 138 opens. Also, the second inflow valve 136 opens and the second outflow valve 140 closes. Medical fluid in the barrel 118 of the first pumping syringe 114 is expelled from the barrel 118 through the open first outflow valve 138 to the patient line as shown by arrow A4 (in FIG. 4D). Simultaneous with the expulsion of the medical fluid from the barrel 118 of the first pumping syringe 114, medical fluid is drawn from the reservoir syringe 112 into the barrel 118 of the second pumping syringe 116 through the open second inflow valve 136 in the direction of arrow A5 (in FIG. 4D).

Once the plungers 120 reach end positions (e.g., the plunger 120 of the first pumping syringe 114 reaches its distal-most position and the plunger 120 of the second pumping syringe 116 reaches its proximal-most position), the direction of movement of the plungers 120 is again reversed. Upon reversing direction, the medical fluid in the barrel 118 of the second pumping syringe 116 is expelled from the second pumping syringe 116 through the second outflow valve 140, as shown by arrow A6 in FIG. 4C. Simultaneous with expelling the medical fluid from the second pumping syringe 116, additional medical fluid is drawn from the reservoir syringe 112 into the barrel 118 of the first pumping syringe 114, as shown by arrow A3 in FIG. 4C. The first and second actuators 128, 130 continue to repeatedly move the plungers 120 simultaneously through the barrels 118 of the pumping syringes 114, 116 in opposite directions for a duration of a medical infusion procedure or infusion event. This repeated movement causes the medical fluid to be expelled from the pump 110 as a continuous or substantially continuous flow, alternating between fluid expelled from the barrel 118 of the first pumping syringe 114 and from the barrel 118 of the second pumping syringe 116.

At step 216, the medical fluid expelled from the pump 110 is delivered through the patient line to the vasculature of the patient. For example, medical fluid can be expelled from the pump 10, 110 through outflow ports 70, 154, 156 and into the patient line 14, 158, 160 for delivery to vasculature of the patient. For the pump 10 with the single pumping syringe 24, the medical fluid can pass through the single pump outflow port 70 to the patient line 14, as a semi-continuous fluid flow occurring when the plunger 32 of the single pumping syringe 24 moves through the barrel 30 in the distal direction (shown by arrow D in FIG. 1C). For the pump 110 with the dual pumping syringes 114, 116, the first patient line 158 extends from the first outflow port 154 and the second patient line 160 extends from the second outflow port 156. The medical fluid is expelled from the pump 110 through the outflow ports 154, 156 to the patient lines 158, 160. As previously described, in some examples, the patient lines 158, 160 are separate along their entire lengths. In that case, fluid expelled from the pump 110 into the first patient line 158 passes through the first patient line 158 to vasculature of the patient at a first location. The medical fluid expelled from the pump 110 into the second patient line 160 passes through the second patient line 160 to the vasculature of the patient at a second location. In other examples, the first and second patient lines 158, 160 connect together at the intersection point 164 forming a common patient line 162. Fluid passing through each of the patient lines 158, 160 passes into the common patient line 162, where it is delivered to the vasculature of the patient as a continuous or substantially continuous fluid flow.

At step 218, the method further comprises removing an empty reservoir syringe 12, 112 from the pump 10, 110 and replacing the reservoir with a new full reservoir syringe 12, 112. In some examples, replacing an empty reservoir syringe 12, 112 can occur while the actuator(s) 50, 128, 130 continues to move the plunger(s) 32, 120 through the barrel(s) 30, 118 of the pumping syringe(s) 24, 114, 116. Accordingly, medical fluid in the pumping syringe(s) 24, 114, 116 can continue to be delivered to the patient even as the reservoir syringe 12, 112 is being replaced. Therefore, it may not be necessary to turn off the pump 10, 110 or actuator(s) 50, 128, 130 while replacing an empty reservoir 12, 112, provided that the replacement can be performed quickly. After the reservoir syringe 12, 112 is replaced with the full reservoir 12, 112, the medical fluid can continue to be delivered to the patient for a remaining duration of the medical infusion procedure or event.

While examples of the pumps, fluid infusion systems, and methods are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

What is claimed is:

1. A pump for delivering fluid from a reservoir to a patient, comprising:
at least one pumping syringe comprising a barrel and a plunger movable through the barrel;
at least one actuator for moving the plunger through the barrel to draw the fluid from the reservoir into the barrel and to expel the fluid from the barrel towards the patient;
at least one inflow valve between the reservoir and the barrel of the at least one pumping syringe that opens when the plunger of the at least one pumping syringe moves to aspirate the fluid from the reservoir into the barrel of the at least one pumping syringe; and
at least one outflow valve between the barrel of the at least one pumping syringe and the patient that opens when the plunger moves to expel the fluid from the barrel of the at least one pumping syringe towards the patient.

2. The pump of claim 1, wherein the pump is a wearable pump worn by an ambulatory patient, and wherein the fluid comprises at least one of saline, a liquid medication, or a total parenteral nutrition liquid.

3. The pump of claim 1, further comprising a valve set connected to the at least one pumping syringe and to the reservoir, the valve set comprising:
a housing enclosing at least one fluid chamber;
at least one inflow port, wherein the fluid passes from the reservoir to the at least one fluid chamber through the at least one inflow port;
at least one syringe port fluidly connected to an interior of the barrel of the at least one pumping syringe, wherein the fluid passes between the interior of the barrel and the at least one fluid chamber through the at least one syringe port; and
at least one outflow port, wherein the fluid passes from the at least one fluid chamber to the patient through the at least one outflow port.

4. The pump of claim 1, wherein the at least one inflow valve comprises an inflow check valve that automatically opens as the fluid is aspirated into the barrel of the at least one pumping syringe, and
wherein the at least one outflow valve comprises an outflow check valve that automatically opens when the fluid is expelled from the barrel of the at least one pumping syringe.

5. The pump of claim 4, wherein a cracking pressure of the inflow check valve and/or the outflow check valve is greater than atmospheric pressure to prevent siphoning of the fluid from the reservoir through the inflow check valve and the outflow check valve.

6. The pump of claim 1, wherein a volume of the barrel of the at least one pumping syringe is from about 3 mL to about 5 mL.

7. The pump of claim 1, further comprising a pump controller electrically coupled to the at least one actuator configured to cause the at least one actuator to repeatedly move the plunger through the barrel of the at least one pumping syringe at a predetermined rate.

8. The pump of claim 7, wherein the pump controller is configured to monitor a torque exerted by the at least one actuator on the plunger to detect when the plunger approaches an end of stroke position, and to change direction of the plunger when the torque increases above a threshold.

9. The pump of claim 7, further comprising at least one position sensor for detecting a position of the plunger within the barrel, wherein the pump controller is configured to cause the at least one actuator to change direction of the plunger based on information detected by the at least one position sensor.

10. The pump of claim 1, further comprising a sealed enclosure that contains at least portions of the at least one pumping syringe, the reservoir, the at least one inflow valve, and the at least one outflow valve, to restrict access to the fluid in the reservoir.

11. The pump of claim 1, wherein the reservoir is removable from other portions of the pump allowing the reservoir to be replaced without ceasing operation of the at least one actuator and the at least one pumping syringe.

12. The pump of claim 1, wherein the at least one pumping syringe comprises a first pumping syringe and a second pumping syringe, and
wherein the at least one actuator comprises a first actuator that moves the plunger of the first pumping syringe and a second actuator that moves the plunger of the second pumping syringe.

13. The pump of claim 12, wherein the fluid expelled from the first pumping syringe passes to a first patient line and fluid expelled from the second pumping syringe passes to a second patient line that is separate from the first patient line.

14. The pump of claim 12, wherein the fluid expelled from the first pumping syringe and the fluid expelled from the second pumping syringe pass to the patient through a common patient line.

15. The pump of claim 12, wherein the at least one inflow valve comprises:
a first inflow valve between the reservoir and the barrel of the first pumping syringe that opens when the plunger of the first pumping syringe moves to aspirate the fluid from the reservoir into the barrel of the first pumping syringe; and
a second inflow valve between the reservoir and the barrel of the second pumping syringe that opens when the plunger of the second pumping syringe moves to aspirate the fluid from the reservoir into the barrel of the second pumping syringe,
wherein the first inflow valve closes when the plunger of the first pumping syringe moves to expel the fluid from the barrel of the first pumping syringe, and the second inflow valve closes when the plunger of the second pumping syringe moves to expel the fluid from the barrel of the second pumping syringe.

16. The pump of claim 12, wherein the at least one outflow valve comprises a first outflow valve between the barrel of the first pumping syringe and the patient that opens when the plunger of the first pumping syringe moves to expel the fluid from the barrel towards the patient, and a second outflow valve between the barrel of the second pumping syringe and the patient that opens when the plunger of the second pumping syringe moves to expel the fluid from the barrel towards the patient.

17. The pump of claim 12, further comprising a valve set connected to the first pumping syringe, the second pumping syringe, and the reservoir, the valve set comprising:
a housing enclosing at least one fluid chamber;
an inflow port, wherein the fluid passes from the reservoir to the at least one fluid chamber through the inflow port;
a first syringe port fluidly connected to an interior of the barrel of the first pumping syringe, wherein the fluid passes between the interior of the barrel of the first pumping syringe and the at least one fluid chamber through the first syringe port;
a second syringe port fluidly connected to an interior of the barrel of the second pumping syringe, wherein the fluid passes between the interior of the barrel of the second pumping syringe and the at least one fluid chamber through the second syringe port;
a first outflow port, wherein the fluid passes from the barrel of the first pumping syringe toward the patient through the first outflow port; and
a second outflow port, wherein the fluid passes from the barrel of the second pumping syringe toward the patient through the second outflow port.

18. The pump of claim 12, further comprising a pump controller electronically coupled to the first actuator and the second actuator, configured to cause the plunger of the first pumping syringe and the second pumping syringe to move substantially simultaneously in opposite directions, which causes fluid to be expelled from the pump toward the patient as a continuous or substantially continuous flow.

19. A fluid infusion system, comprising:
the pump of claim 1;
the reservoir fluidly connected to the pump containing the fluid to be delivered to the patient; and
at least one patient line fluidly connected to the barrel of the at least one pumping syringe at a position distal to the at least one outflow valve, wherein the patient line is configured to deliver the fluid from the pump to vasculature of the patient.

20. A method for fluid infusion to a patient, the method comprising:
attaching the pump of claim 1 to the reservoir, such that the reservoir is in fluid communication with the barrel of the at least one pumping syringe;
placing the pump in fluid communication with vasculature of the patient; and
causing the at least one actuator of the pump to repeatedly move the plunger through the barrel of the at least one pumping syringe thereby causing the fluid to pass from the reservoir into the barrel of the at least one pumping syringe and from the barrel of the at least one pumping syringe to the patient.
